(19) United States
(12) Patent Application Publication
(10) Pub. No.: US 2011/0092493 A1
(43) Pub. Date: Apr. 21, 2011

(54) Dose-controlled transdermal promethazine compositions and methods of use

(76) Inventors: Clark Levi, (US); Susan M. Welsh, (US); Kenneth V. Phelps, (US); Frank G. Pilkiewicz, (US)

(21) Appl. No.: 12/284,671

(22) Filed: Sep. 24, 2008

(51) Int. Cl. A61K 31/5415; A61P 1/08

(52) U.S. Cl. 514/226.2

(57) ABSTRACT

The current invention provides formulations for transdermal delivery of promethazine which achieve delivery of the drug with consistent plasma levels. The topical formulations of the invention are superior to both rectal suppositories and oral dosage forms in that variable blood levels, first-pass metabolism, unpredictable peaks in blood levels, and variable bioavailability are minimized. Formulations of the invention provide antiemetic and antipruritic relief to patients in need of treatment while minimizing side effects and adverse reactions known to occur with other routes of administration and other formulations.

### BACKGROUND OF THE INVENTION

Promethazine is a versatile drug with antihistaminic, anticholinergic, sedative, and antiemetic effects. Promethazine was introduced in 1946 and has since been used in a variety of clinical situations including prevention and treatment of nausea and vomiting caused by narcotic therapy, migraines, and chemotherapy, as a sedative for preoperative drug regimens and during labor, as well as for motion sickness (Strenkoski-Nix, L. et al, 2000, Am. J. Health Syst. Pharm., 57:1499-1505). Promethazine hydrochloride is a widely used product that has been marketed for many decades in various dosage forms (Federal Register. 2002, 167). Currently, it is predominantly used for its antiemetic effects (Migraine Awareness Group: A National Understanding for Migraineurs. Phenergan for Migraines. 1999). The intravenous administration of promethazine is associated with the risk of debilitating side effects. The drug is a known vesicant which is highly caustic to the intimae of blood vessels and surrounding tissue. Formulated with phenol, promethazine has a pH between 4 and 5.5. Although deep intramuscular injection into a large muscle is the preferred parenteral route of administration, product labeling states that the drug may be given by slow IV push, which is how it is typically given in most hospitals. Severe, tragic, local injuries after infiltration or inadvertent intra-arterial injections has caused adverse effects including burning, erythema, pain, swelling, severe spasm of vessels, thrombophlebitis, venous thrombosis, phlebitis, nerve damage, paralysis, abscess, tissue necrosis, and gangrene. Sometimes surgical intervention has been required, including fasciotomy, skin graft, and even amputation. (Paparella, S., 2006, J. Emergency Nursing 33(1):53-55). Promethazine has been reported to be an economic and effective option for treatment of nausea and vomiting in comparison with other treatments (Wright, C. et al, 1998, Yale J. Biol. Med., 1:391-395). Although no approved topical or transdermal formulations are currently available in the US, compounding pharmacies have provided custom promethazine formulated in a pleuronic lecithin organogel (PLO gel) (U.S. Pharmacist 1999 74-5; Lloyd, A., 2001, Int. J. Pharm. Compound., 5(1):51, Lloyd, V., 1999, Allen's Compounded Formulations: The United States Pharmacist Collection, 1995-1998, Washington D.C.: American Pharmaceutical Association). In the clinical setting a patient with post operative nausea and vomiting (PONV) is best treated using a route of administration which avoids the gastrointestinal tract. Therefore, a topically applied transdermal route of administration, which avoids invasive procedures such as intravenous therapy, represents an optimal mode of delivery. The present invention provides formulations for topically applied transdermal administration of promethazine.

Several studies have evaluated the pharmacokinetics of promethazine hydrochloride administered by different routes. In a study comparing a 50 mg suppository with oral syrup (50 mg), absorption of both promethazine formulations exhibited pronounced variability, with coefficients of variation greater than 84% for the area under the plasma concentration-versus-time curve (AUC) (Stavchansky S. et al, 1987, J Pharm. Sci., 76:441-5). The mean AUC from 0 to 24 hours (AUCO-24) for the suppository was 168 ng·hr/mL, compared with 268 ng·hr/mL for the oral syrup. Another study compared a generic promethazine hydrochloride 50 mg suppository with a brand-name preparation of promethazine hydrochloride oral syrup (50 mg), and with a brand-name 50 mg suppository (Schwinghammer T. et al, 1984, Biopharm. Drug Dispos., 5(2):185-94). Both suppository treatments yielded a lower maximum promethazine concentration and a longer time to maximum promethazine concentration than did the oral syrup. However, there were no significant differences in AUC₀₋₂₄. Both suppository treatments and the oral syrup demonstrated high variability with respect to promethazine pharmacokinetics. The pharmacokinetics of intravenous and oral promethazine have also been compared (Taylor G. et al, 1983, Br. J. Clin. Pharmacol., 15:287-93). On average, 88% of a promethazine dose is absorbed after oral administration; however, the absolute bioavailability is only 25% because of first-pass clearance. This indicates that transdermal administration, which avoids first-pass metabolism, is a superior mode of delivering promethazine to patients in need. Another study showed that the pharmacokinetics of promethazine administered in oral syrup and three rectal suppository treatments were highly variable (Strenkoski-Nix, L. et al, 2000, supra). In an open, four-way cross-over design, 36 subjects received 50 mg promethazine syrup, one 25 mg suppository, two 12.5 mg suppositories, or one 50 mg suppository. After the 50 mg oral dose, the maximum blood concentration (Cmax) ranged from 6.4 to 54 ng/ml. Cmax for the 50 mg suppository was lower but varied from 1.0 to 3.4 ng/ml. In general, the suppositories produced a lower Cmax and later time at which maximum blood concentration (tmax) was achieved than the syrup. All formulations were comparable in terms of dose-normalized AUC and elimination half-life (t½), and the three suppository treatments were comparable in terms of dose-normalized Cmax. The mean relative bioavailability for the three suppository treatments ranged from 70% to 97%. The large variability of promethazine levels arising from oral or rectal dosing is demonstrated by the individual relative bioavailabilities found in this study. Individual relative bioavailabilities (reference bioavailability [100%]=50 mg [10 mL] of promethazine oral syrup) ranged from 4% to 343%. The most common adverse events were somnolence (35%) and headache (28%), with somnolence being most frequent with the 50 mg oral (22%) followed by 50 mg suppository (17%) and then with the 25 mg suppository (5%). The plasma levels were variable and the adverse events appeared dose-related. In another study, promethazine 50 mg applied to the wrist of 15 healthy volunteers in a topical pluronic lecithin organogel resulted in drowsiness in 73% of subjects and sedation in 50% of subjects (Glisson J. et al, 2005, International Journal of Pharmaceutical Compounding, 9). In this study, dosage of promethazine was not controlled, and a high percentage of patients experienced unpleasant side effects caused by high blood levels of the drug. Topical promethazine used as an antipruritic agent has been associated with various safety concerns including neurologic toxicity syndrome, and skin sensitization (Shawn D. et al, 1984, Can. Med. Assoc. J., 130[11]:1460-1461; Pan V Anuncibay P. et al, 1989, DICP, The Annals of Pharmacotherapy, 23:89). Thus, current modes of delivery of promethazine suffer from a highly variable absorption, widely unpredictable bioavailability, and adverse events linked to uncontrolled blood levels of the drug. In addition, poorly controlled blood levels of promethazine render current modes of administration undesirable for its use in the prophylaxis of motion sickness in some cases. For example, orally dosed promethazine is used with particular caution in astronauts and is typically not permitted in pilots (Cosings P. et al, 2000 Aviat. Space Environ. Med. 71(10):1013-22).

### SUMMARY OF THE INVENTION

The current invention provides formulations for transdermal delivery of promethazine which achieve delivery of the drug with consistent plasma levels. The topical formulations of the invention are superior to both rectal suppositories and oral dosage forms in that variable blood levels, first-pass metabolism, unpredictable peaks in blood levels, and variable bioavailability are minimized. Formulations of the invention provide antiemetic and antipruritic relief to patients in need of treatment, while minimizing side effects and adverse reactions known to occur with other routes of administration and other formulations.

### DETAILED DESCRIPTION OF THE INVENTION

As used throughout the description of the invention, the following terms, unless otherwise indicated, shall be understood to have the following meanings:

“Dose” means the amount of drug delivered to the blood stream.

“Patient” means a mammal including a human.

“Therapeutically effective dose” means an amount of promethazine effective for treating post-operative nausea and vomiting, pain, and pruritus, thus producing the desired therapeutic effect, such amount determined by practitioners with skill in the art.

“Treat” or “treatment” or “treating” mean to lessen, eliminate, inhibit, improve, alter, or prevent a disease or condition by topically applied transdermal administration of promethazine formulations of the present invention.

“Pharmaceutically acceptable” means compounds, methods, procedures, formulations, addition salts, solutions, preparations, and routes of administration known by those with skill in the art to be proper and safe in the practice of administering biologically active medicaments to patients.

“Transdermal composition” means a cream, gel, ointment, lotion, levigate, solution, paste, bioadhesive, salve, milk, impregnated pad, spray, suspension, foam, or the like, containing an active drug which is applied to body surfaces such as the skin so that the active drug passes through the skin and enters the peripheral circulation.

“Preservative” means a compound which preserves, protects, or otherwise stabilizes one or more components of the formulation by virtue of its characteristics including, but not limited to, antimicrobial activity, anti-oxidant activity, and chemical stability.

“Carrier” means a compound or mixture of compounds into which the active drug and excipients of the invention are dissolved, suspended, levigated, intermixed, homogenized, or emulsified to provide a composition of the invention.

“Oil phase” means one or more water-insoluble hydrophobic components of the formulation which participate in an emulsion or organogel composition.

“Transdermal penetration enhancers” mean compounds chosen for the specific function of altering the physical characteristics of the skin in order to facilitate permeation of chemical substances through the skin and into the blood system.

“Emulsifying agents” mean compounds selected for the specific function of allowing components of the formulations of the invention to form stable emulsions without interfering with the express designed properties of the formulations as disclosed herein.

“Emulsion stabilizers” are surface-active agents contain both hydrophobic groups and hydrophilic groups used in formulation and stabilization of emulsions.

“Buffering agents” are compounds or mixtures of compounds which impart a known and constant pH to formulations containing water, or compounds or mixtures of compounds which impart a known and constant apparent pH to anhydrous formulations.

“Liposomes” are emulsions, foams, micelles, insoluble monolayers, liquid crystals, phospholipid dispersions, lamellar layers and spherical vesicles comprising a lipid bilayer membrane, which can be used to encapsulate an active pharmaceutical ingredient to enhance percutaneous drug delivery.

“Lipid complexes” are self-assembled aggregations of lipid molecules into which active pharmaceutical ingredients are inserted to enhance percutaneous drug delivery.

“Biocompatible polymeric matrices” are synthetic polymers designed to entrap an active pharmaceutical ingredient which may subsequently diffuse away, to enhance percutaneous drug delivery.

“Microspheres” are spherical particles composed of various natural and synthetic materials with diameters in the micrometer range which can be used to encapsulate an active pharmaceutical ingredient to enhance percutaneous drug delivery.

“Unit-dose pack” means a container, tube, capsule, vial, ampoule, syringe, envelope, bladder, bag, cartridge, cassette, or the like designed to contain a single dose of a transdermal promethazine formulation to be applied on selected skin surfaces at prescribed intervals by a patient or health care professional in order to control the dose of drug received.

A “transdermal patch” is a medicated adhesive patch that is placed on the skin to deliver a specific dose of medication through the skin and into the bloodstream.

A “transdermal device” as used herein means an article of manufacture designed to facilitate the transcutaneous permeation of an active pharmaceutical ingredient when contacting the skin of a patient and includes solid, liquid, semi-solid or gel formulations comprising at least one reservoir and an adhesive membrane, iontophoretic devices (which utilize electricity to facilitate transcutaneous permeation), and phonophoretic devices (which utilize ultrasound energy to facilitate transcutaneous permeation).

It is understood that, throughout the discussion, that “%” and “percent” mean percent by weight (% w/w), unless otherwise indicated.

The typical doses of promethazine administered to patients by injection or suppository is 25 mg, a level which produces sedation. Because of this, and the relatively low bioavailability of an oral dose, also typically 25 mg, it is considered that formulations containing a lower dose of promethazine can be applied topically for transdermal delivery for optimum therapeutic effect. (Phenergan Package Insert, Philadelphia, Pa.: Wyeth; December 2004). In current usage, topical promethazine compounded formulations which contain high concentrations of the drug are applied to the skin. This may result in the administration to the patient of a dose of drug in large excess over the therapeutic dose. It is therefore one aspect of the invention to provide transdermal promethazine formulations which deliver significantly less drug to the patient. In some embodiments, transdermal formulations with a lower concentration of promethazine are provided. In other embodiments, a unit-dose pack is provided wherein the amount of promethazine applied topically for transdermal absorption is controlled by the concentration and volume of the package, thus preventing the accidental administration of excessive amounts of drug, and thus avoiding side effects and adverse reactions arising from blood levels of drug in excess of the therapeutic value.

The present invention provides formulations for topically applied transdermal delivery of promethazine which are an improvement over currently used modalities of administration. The oral bioavailability of promethazine is low, averaging 22% to 25% (Ramanathan, R. et al 1998; Pharmacological Research 38:35-39). As opposed to oral formulations (for example Phenergan® Syrup Fortis, which contains five mg per mL promethazine, and which also contains glycerin: saccharin sodium, sodium benzoate, sodium citrate, sodium propionate, and water) and suppository formulations (for example Phenergan® suppositories which may contain 25 mg promethazine per suppository, and which also contain ascorbyl palmitate, silicon dioxide, white wax, and cocoa butter) a composition of one embodiment of the present invention can achieve rapid percutaneous delivery of promethazine and maintenance of promethazine blood levels below that which causes side effects or adverse reactions. Peak plasma levels following therapeutic oral doses of 30 to 50 mg in adults have ranged from 11 to 23 ng/mL (Wallace et al. 1981; Clin. Chem. 27:253-255). Adverse effects following intramuscular injection were associated with plasma levels of 48 ng/mL (Schwinghammer et al. 1984; Biopharmaceut. Drug Disp., 5:185-194). In an example of an embodiment of the invention, a formulation comprising a range of about 1% to about 1.5% promethazine, and a range of about 5% to about 10% 1-dodecylazacycloheptan-2-one, is provided in a 1.0 mL unit dose pack, which limits the possibility that excessive blood levels associated with adverse effects through the control of the strength and volume of the formulation. This is to be compared to a composition offered by Novartis Consumer Health in tubes of 30 g. which is composed of: promethazine base 2% in a scented, non-greasy water miscible base, fragrance, glyceryl monostearate, lanolin, methylparaben, propylparaben, stearyl alcohol, stearic acid, triethanolamine and water. This latter composition may be purposely or inadvertently abused by application of an excess amount of cream to the skin of the patient. Alternatively, the application of an insufficient volume of this medicament results in the failure to achieve effective therapeutic blood levels. In contrast, application of the formulation contained in a single unit dose pack provides sufficient drug and permeation enhancer to deliver a therapeutic dose while minimizing the risk of elevated blood drug levels.

It is an object of the invention to provide topically applied transdermal formulations of promethazine which furnish rapid onset of action, high bioavailability, consistent therapeutic, but sub-toxic, blood levels, with low variability over the course of treatment.

In one embodiment, a formulation of the invention comprises promethazine mixed with a suitable carrier.

In another embodiment, a formulation of the invention further comprises excipients such as emulsifying agents.

In another embodiment, a formulation of the invention further comprises excipients such as buffering agents.

In another embodiment, a formulation of the invention further comprises excipients such as preservatives.

In another embodiment, a formulation of the invention further comprises excipients such as transdermal penetration enhancers.

In another embodiment of the invention, a formulation of the invention comprises liposomes.

In another embodiment of the invention, a formulation of the invention comprises lipid complexes.

In another embodiment of the invention, the amount of a formulation of the invention administered to a patient is controlled by the use of unit-dose packages.

In another embodiment of the invention, a formulation of the invention is delivered through the use of a transdermal device.

In another embodiment of the invention, a formulation of the invention is delivered through the use of a transdermal patch.

Promethazine is commercially available (LGM Pharmaceuticals, Inc. Boca Raton, Fla.; Zhejiang Medicines and Health Products Import and Export Co., Ltd., Hangzhou, China; Voigt Global Distribution Inc., Lawrence, Kans.) In some embodiments, formulations of the invention comprise about 0.1% to about 10% promethazine. In some embodiments, formulations of the invention comprise about 1% to about 10% promethazine. In some embodiments, formulations of the invention comprise about 1%, about 2%, about 3%, about 4%, about 5%, about 6%, about 7%, about 8%, about 9% or about 10% promethazine.

Suitable carriers and carrier components for topically applied formulations include those described in reference works such as Martindale—The Extra Pharmacopoeia, Pharmaceutical Press, London, 1993. For example, carriers suitable for use in compositions according to the invention include, but are not limited to, glycerin, glycerol, propylene glycol, hexylene glycol, isopropyl myristate, gelatin, lecithin, urea, carnauba wax, Brij 72, stearate NF, polysorbate 60, polyglyceryl-3-oleate, sorbitol solution (USP), microcrystalline wax, IGI Microsere, white petrolatum, xanthen gum, cetomacrogol 1000 BP, cetostearyl alcohol, PEG, cyclomethicone, demethiconol, dimethicone copolyol, hydroxyoctacosanyl hydroxy stearate, methoxy PEG-22/dodecylglycol copolymer, Carbomer 940 NF, docusate sodium, trolamine NF, Carbomer 934P, polyoxomer 407, triolein, egg yolk phospholipids, cold cream (USP), hydrophilic ointment (USP), an emulsion of mineral oil and purified water, purified water, and mixtures thereof, or their equivalents. In some embodiments of the invention, emulsions comprising one or more hydrophobic carrier component (“oil phase”) and purified water are provided. In some embodiments, emulsion formulations of the invention comprise from 1% to 40% oil phase. In some embodiments, emulsion formulations of the invention comprise from 1% to 10% oil phase. In some embodiments, formulations of the invention comprise about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, or about 35% oil phase.

In some embodiments, emulsion formulations of the invention further comprise one or more emulsifying agents. In some embodiments, the emulsifying agent is about 1% to about 10% of the formulation. Emulsifying agents include, but are not limited to, propylene glycol, Span 60 (sorbitan monostearate, ICI Americas, Inc.), Span 80 (sorbitan monooleate, ICI Americas, Inc.), non-ionic emulsifiers like polyoxyethylene oleyl ether, PEG-40 stearate, ceteareth-12 (e.g. Eumulgin B-1 manufactured by Henkel), ceteareth-20 (e.g., Eumulgin B-2 manufactured by Henkel), ceteareth-30, Lanette O (manufactured by Henkel; ceteareth alcohol), glyceryl stearate (e.g., Cutina GMS manufactured by Henkel), PEG-100 stearate, Arlacel 165 (glyceryl stearate and PEG-100 stearate), steareth-2 and steareth-20, cationic emulsifiers like stearamidopropyl dimethylamine and behentrimonium methosulfate, nonionic surfactants like Surfactant 190 (dimethicone copolyol), Polysorbate 20 (Tween 20, Uniqema/ICI), Polysorbate 40 (Tween 40, Uniqema/ICI), Polysorbate 60 (Tween 60, Uniqema/ICI), Polysorbate 80 (Tween 80, Uniqema/ICI), lauramide DEA, cocamide DEA, and cocamide MEA, amphoteric surfactants like oleyl betaine and cocamidopropyl betaine (Velvetex BK-35), and cationic surfactants like Phospholipid PTC (Cocamidopropyl phosphatidyl PG-dimonium chloride), Pemulen TR 1, Pemulen TR 2, Carbopol 1342, Carbopol 1382, Carbomer 1342, Carbomer 934, Carbomer 934P, Carbomer 940, Carbomer 941, Carbomer 974P, Carbomer 980, and Carbomer 981, or their equivalents. It is recognized that a mixture of two or more emulsifying agents may be selected to achieve a formulation possessing the desired parameters. In some embodiments, the formulations of the invention contain about 1%, about 2%, about 3%, about 4%, about 5%, about 6%, about 7%, about 8%, about 9% or about 10% emulsifying agents.

In some embodiments, emulsion formulations of the invention further comprise one or more emulsion stabilizer. In some embodiments, the emulsion formulations of the invention comprise from 0.1% to 5% emulsion stabilizer. Emulsion stabilizers of the invention include, but are not limited to, Pemulen TR-2I (Lubrizol Advanced Materials, Inc.), and Carbopol 981 (Lubrizol Advanced Materials, Inc.), mannide monooleate (ARLACEL A™, ICI), dextran 70,000, polyoxyethylene ethers (Triton X™ series, Dow Chemical), polyglycol ethers (Tergitol™, Dow Chemical), straight-chain or branched-chain saturated or unsaturated fatty acids of 6-22 carbon atoms, stearic acid, oleic acid, linoleic acid, palmitic acid, linoleic acid, myristic acid and their salts, aliphatic primary amines or aliphatic secondary amines containing 2-22 carbon atoms, ethanolamine, propylamine, octylamine, stearylamine, oleylamine, basic amino acids such as lysine, histidine, ornithine, arginine, sterols such as cholesterol, cholestanol, phosphatidic acids, and gangliosides, or their equivalents. In some embodiments, the formulations of the invention further comprise about 1%, about 2%, about 3%, about 4%, or about 5% emulsion stabilizer.

In some embodiments, formulations of the invention further comprise on or more buffering agents in order to substantially control the pH or the apparent pH of the composition. Buffering agents are well-known in the art. Buffering agents of the invention include, but are not limited to, those derived from the alkali or alkaline earth salts of acetic, aconitic, boric, citric, glutaric, lactic, malic, succinic, phosphate and carbonic acids; naturally-occurring alpha-amino acids and pharmaceutically acceptable salts thereof; organic buffers such as HEPES (N-2-hydroxyethylpiperazine-N′-2-ethanesulfonic acid), BES (N,N-bis[2-hydroxyethyl]-2-amino-ethanesulfonic acid), TES (N-tris[Hydroxymethyl]methyl-2-aminoethanesulfonic acid), MOPS (morpholine propanesulphonic acid), PIPES (piperazine-N,N′-bis[2-ethane-sulfonic acid]), or MES (2-morpholino ethanesulphonic acid), TRIS (tris(hydroxymethyl)aminomethane, tricine (N-(Tri(hydroxymethyl)methyl)glycine), bicine (N,N-Bis(2-hydroxyethyl)glycine), Bis-Tris HCl (Bis(2-hydroxyethyl)aminotris(hydroxymethyl)methane, Bis-Tris-propane (1,3-Bis[tris(hydroxymethyl)methylamino]propane), CABS (4-(Cyclohexylamino)-1-butanesulfonic acid), CAPS (3-(Cyclohexylamino)-1-propanesulfonic acid), CAPSO (3-(Cyclohexylamino)-2-hydroxy-1-propanesulfonic acid), CHES (2-(Cyclohexylamino)ethanesulfonic acid), EPPS (N-(2-Hydroxyethyl)piperazine-N′-(3-propanesulfonic acid), HEPBS (N-(2-Hydroxyethyl)piperazine-N′-(4-butanesulfonic acid), TABS (N-tris(Hydroxymethyl)methyl-4-aminobutanesulfonic acid), TAPS (N-[Tris(hydroxymethyl)methyl]-3-aminopropanesulfonic acid), TAPSO (N-[Tris(hydroxymethyl)methyl]-3-amino-2-hydroxypropanesulfonic acid), tromethamine (tris(hydroxymethyl)methylamine), EDTA (ethylenediaminetetraacetic acid), DTPA (diethylaminetriaminepentaacetic acid), and triethanolamine, and pharmaceutically acceptable salts thereof, and their equivalents. In some embodiments, formulations of the invention further comprise one or more buffering agents to provide a composition with pH of about 2 to a pH of about 8. In some embodiments, formulations of the invention further comprise one or more buffering agents to provide a composition with pH of about 3 to a pH of about 7. In some embodiments, formulations of the invention further comprise one or more buffering agents to provide a composition with pH of about 4. In some embodiments, formulations of the invention further comprise one or more buffering agents to provide a composition with pH of about 5. In some embodiments, formulations of the invention further comprise one or more buffering agents to provide a composition with pH of about 6. In some embodiments, formulations of the invention further comprise one or more buffering agents to provide o composition with pH of about 7.

In some embodiments, formulations of the invention further comprise one or more transdermal permeation enhancers. It is well-known that, for certain chemical substances, controlled-voltage electricity, and ultrasound can individually alter some barrier properties of human skin and, when properly utilized, can enhance the penetration of active pharmaceutical intermediates through the dermal barrier. Chemical transdermal permeation enhancers have been reviewed (Williams, A. et al, 2004, Adv. Drug Deliv. Rev., 56: 603-618; Büyüktimkin, N. et al, 1997, In: Ghosh, T. K., Pfister, W. R. eds., Transdermal and Topical Drug Delivery Systems, Interpharm Press, Buffalo Grove, Ill., pp. 357-476). Transdermal permeation enhancers of the invention include, but are not limited to, Azone (1-dodecylazacycloheptan-2-one), diethyl sebacate, (International Patent Publication No. WO 92/19271), lauric acid diethanolamide (Japanese Patent Laid-Open No. 11-335281), N-methyl-2-pyrrolidone (Japanese Patent Laid-Open No. 8-113533), lauroyl macrogol-6 glyceride (International Patent Publication No. 95/28932, U.S. Pat. No. 5,503,843), propylene glycol monolaurate (Japanese Patent Laid-Open No. 8-40937, U.S. Pat. No. 5,059,426, U.S. Pat. No. 5,053,227, U.S. Pat. No. 4,973,468, U.S. Pat. No. 5,006,342, U.S. Pat. No. 4,906,463, U.S. Pat. No. 5,006,342), triacetin (Japanese Patent Laid-Open No. 2001-39865, U.S. Pat. No. 5,601,839), isopropyl myristate and isopropyl palmitate (Japanese Patent Laid-Open No. 2001-131089, Japanese Patent Laid-Open No. 8-225448), and 1-menthol (Japanese Patent Laid-Open No. 2000-119195), Transkarbam 12 (Hrabálek, A. et al, 2006, Pharm. Res., 23(5):912-919), fatty acids, fatty acid esters, alpha-tocopherol, glycols, glycol monoethers, glycol diethers, dimethylsulphoxide, caprolactam, dimethylisosorbide, isopropylidene glycerol, dimethylimidazolidinone, ethyl lactate, the polyoxyethylenated C₈-C₁₀ glycerides, polyethylene glycol 20 glyceryl laurate and dimethylacetamide, terpinolene, α-phellandrene, ocimene, myrcene, (1R)-(−)-myrtenal, (S)-(−)-perillaldehyde, carvacrol, thymol, (R)-(−)-carvone, (1R)-(−)-myrtenol, (−)-α-thujone, (R)-(+)-pulegone, (+)-dihydrocarvone, (−)-carveol, citral, (−)-isopulegol, (+)-dihydrocarveol, (−)-dihydrocarveol, (S)-(−)-citronellal, geraniol, nerol, (±)-linalool, menthone, β-citronellol, L-(−)-menthol, cyclohexanemethanol, A-humulene, (−)-α-cedrene, (+)-β-cedrene, (+)-aromadendrene, (+)-longifolene, (−)-trans-caryophyllene, (−)-caryophyllene oxide, (−)-epiglobulol, (−)-guaiol, (+)-cedrol, (−)-isolongifolol, (−)-α-santonin, octisalate, (+)-cedryl acetate, retinol, phytol, squalene, (±)-α-bisabolol, farnesol, (±)-nerolidol, eucarvone, retinoic acid, and β-carotene.

In some embodiments, formulations of the invention further comprise one or more preservatives. In topically applied formulations, a preservative is a compound which preserves, protects, or otherwise stabilizes one or more components of the formulation by virtue of its characteristics including, but not limited to, antimicrobial activity, anti-oxidant activity, and chemical stability. Preservatives of the invention include, but are not limited to, chloro-m-cresol, citric acid, disodium edetate, ethoxylated alcohol, glycerin, 1,2,6-hexanetriol, methylparaben, parabens, potassium sorbate, propyl gallate, propylene glycol, propylparaben, sodium bisulfite, sodium citrate, butylparaben, sodium metabisulfite, sorbic acid, tannic acid, zinc stearate, butylated hydroxytoluene, butylated hydroxyanisole, benzoic acid, salicylic acid, propylparaben, dichlorobenzyl alcohol, formaldehyde, alpha-tocopherol, sodium ascorbate, ascorbic acid, ascorbyl palmitate phenol, m-cresol, bisphenol, cetrimide, benzalkonium chloride, sorbic acid, polyquaternum-1, chlorobutanol, chlorhexidine, Dowcell 200 (Dow Chemical Co., Midland, Mich.), Glydant (dimethylol-25,5-dimethylhydantoin, Lonza, Inc, Fairlawn, N.J.), Germal 115 (imidazolidylurea, Sutton Laboratories, Chatham, N.J.), Germal II (diazolidinylurea, Sutton), sodium hydroxymethyiglycinate, Buzan 1504 (dimethhydroxymethyl pyrazole, Buckman Labs, Memphis, Tenn.), phenoxyethanol, benzoyl peroxide and their equivalents. In some embodiments, the formulations of the invention further comprise from about 0.05% to about 1% preservatives. In some embodiments, the formulations of the invention further comprise about 0.1%, about 0.2%, about 0.3%, about 0.4%, or about 0.5% preservatives.

In some embodiments, formulations of the invention comprise amphipaths, or dual character molecules (polar:nonpolar) that exist as aggregates in aqueous solution. Amphipaths include nonpolar lipids, polar lipids, mono- and diglycerides, sulfatides, lysolecithin, phospholipids, sapchin, bile acids, and salts. These molecules can exist as emulsions and foams, micelles, insoluble monolayers, liquid crystals, phospholipid dispersions and lamellar layers (collectively referred to herein as liposomes). Liposomes are microscopic vesicles having a lipid wall comprising a lipid bilayer, and can be used as drug delivery systems herein as well. Generally, liposome formulations are preferred for poorly soluble or insoluble pharmaceutical agents. Liposomal preparations for use in the instant invention include cationic (positively charged), anionic (negatively charged) and neutral preparations. Cationic and anionic liposomes are readily available. or can be easily prepared using readily available materials such as materials include phosphatidyl choline, cholesterol, phosphatidyl ethanolamine, dioleoylphosphatidyl choline (DOPC), dioleoylphosphatidyl glycerol (DOPG), diopalmitoylphosphatidyl choline (DPPC), dipalmitoylphosphatidyl glycerol (DPPG), dioleoylphoshatidyl ethanolamine (DOPE), cholesterylhemisuccinate (CHEMS), among others. Methods for making liposomes using these materials are well known in the art. In some embodiments, formulations of the invention comprise liposomes. In some embodiments, formulations of the invention comprise lipid complexes. In these formulations, liposomes and lipid complexes are employed to aid in the sustained release and targeting of promethazine. Liposomes and lipid complexes are well known in formulations for pharmaceutical and their use in transdermal delivery has been recognized. (U.S. Pat. No. 5,858,397 to Madden, T. et al). The choice of lipid components and aqueous components impart lipid formulations which allow the controlled diffusion of the drug away from the vesicle and through the skin. Lipid mixtures, for example, but not limited to, dipalmatoylphosphatidylethanolamine and cholesterylhemisuccinate, phosphatidyl ethanolamine and dioleoylphoshatidyl ethanolamine, and dioleoylphosphatidyl choline, dioleoylphosphatidyl glycerol, are useful in imparting this function. Embodiments of the promethazine transdermal liposomal formulation have the following properties:

It can be either a liposome or lipid complex in structure and the promethazine can be located either inside or outside the liposome membrane or lipid complex.

The promethazine liposome or lipid complex can be multi-lamellar vesicles, freeze and thawed multilamellar vesicles, stable plurilamellar vesicles, ethanol infused vesicles or rotary evaporated vesicles as well as lipid complexes, formed via these and other techniques, well-known in the art to form liposomes and lipid complexes.

The lipids used to construct the promethazine liposome or lipid complex can be naturally occurring, semi-synthetic, or synthetic in nature. These lipids can be saturated or unsaturated, neutral, negative, or positively charged.

The lipids used to construct the promethazine liposome or lipid complex can be a phospholipid or steroid.

The lipids used to construct the promethazine liposome or lipid complex can include phospholipids that are lecithins, cholines, derivatized glycerol, derivatized serine, derivatized inositol or derivatized diethanolamines.

The lipids used to construct the promethazine liposome or lipid complex can be steroids such as cholesterol or tocopherol.

The promethazine liposome or lipid complex can be composed of a single lipid or mixtures of two or more lipids in molar ratios that can vary from 0.01% to 100%.

The particle size of the promethazine liposome or lipid complex formulation can be between 40 nanometers to 5 microns in size.

When the liposomes containing promethazine are brought into the proximity of the affected cells, they may deliver the drug by passive diffusion, active diffusion or by any endogenous or exogenous process which serves to facilitate delivery to the required site of action. The promethazine liposome or lipid complex formulation may be designed as such as to effect either a temperature, pH or time-dependent phase transition to occur in the formulation, and so as to cause a release of the promethazine from the formulation via transdermal delivery.

The promethazine liposome or lipid complex formulation may be designed as such as to cause either a hypo- or hyper-osmotic effect to occur so as to cause a release of the promethazine from the formulation via transdermal delivery. The molar ratio of promethazine:lipid in liposomal formulations of the invention range from 1:500 to 10:1.

The preparation of suitable liposomes and methods for coupling them to targeting agents are known in the art and are described, for example, in U.S. Pat. Nos. 4,427,649, 4,957,773, 4,603,044, 4,501,728, 4,037,028 and 4,235,871, 5,043,165, 5,714,163, 5,744,158, 5,783,211, 5,795,589, 5,795,987, 5,798,348, 5,811,118, 5,820,848, 5,834,016 and 5,882,678; Cullis R. et al, 1986, Chem. Phys. Lipids, 40(2-4):127-44). Bangham's procedure produces ordinary multilamellar vesicles (MLVs) (Bangham A. et al, 1965, J. Mol. Biol., August 13, (1):238-52). Lank et al. (U.S. Pat. Nos. 4,522,803, 5,030,453 and 5,169,637), Fountain et al. (U.S. Pat. No. 4,588,578), and Cullis et al. (U.S. Pat. No. 4,975,282) disclose methods for producing multilamellar liposomes having substantially equal interlamellar solute distribution in each of their aqueous compartments. Paphadjopoulos et al. (U.S. Pat. No. 4,235,871) discloses preparation of oligolamellar liposomes by reverse phase evaporation. Unilamellar vesicles can be produced from MLVs by a number of techniques, for example, the extrusion of Cullis et al. (U.S. Pat. No. 5,008,050) and Loughrey et al. (U.S. Pat. No. 5,059,421)). Sonication and homogenization can be so used to produce smaller unilamellar liposomes from larger liposomes (Papahadjopoulos D, et al, 1967; Biochim. Biophys. Acta, 135(4):639-52; Deamer D. et al, 1983; Proc. Natl. Acad. Sci. USA, 80(1):165-8; Chapman D. et al, 1968; Biochim. Biophys. Acta, 163(2):255-61).

The original liposome preparation (Bangham et al, 1965, supra) involves suspending phospholipids in an organic solvent which is then evaporated to dryness leaving a phospholipid film on the reaction vessel. Next, an appropriate amount of aqueous phase is added, the mixture is allowed to “swell”, and the resulting liposomes which consist of multilamellar vesicles (MLVs) are dispersed by mechanical means. This preparation provides the basis for the development of the small sonicated unilamellar vesicles described by (Papahadjopoulos et al, 1967, supra) and large unilamellar vesicles. Techniques for producing large unilamellar vesicles (LUVs), such as reverse phase evaporation, infusion procedures, and detergent dilution, can be used to produce liposomes. A review of these, and other methods, for producing liposomes may be found in “Liposomes”, (Marc Ostro, ed., Marcel Dekker, Inc., New York, 1983, Chapter 1; see also Olson F. et al, 1979; Biochim. Biophys. Acta, 557(1):9-23).

Other techniques that are used to prepare vesicles include those that form reverse-phase evaporation vesicles (REV) (Papahadjopoulos et al., U.S. Pat. No. 4,235,871). Another class of liposomes that may be used are those characterized as having substantially equal lamellar solute distribution. This class of liposomes is denominated as stable plurilamellar vesicles (SPLV) as defined in U.S. Pat. No. 4,522,803 to Lenk, et al. and includes monophasic vesicles as described in U.S. Pat. No. 4,588,578 to Fountain et al. and frozen and thawed multilamellar vesicles (FATMLV) as described above.

In some embodiments, the formulation will be contained in a sealed unit dose package referred to as a “unit dose pack”. According to such embodiments of the invention, the concentration and volume of the promethazine formulation provided the unit dose pack is such that, upon application of the entire contents of a single unit-dose pack, the dose of drug is controlled to limit overdosing. Practitioners of the invention may select unit a dose pack of the invention containing a concentration of promethazine calculated to deliver transdermally a specified dose. In some embodiments, the dose delivered transdermally into the blood by a unit dose pack of the invention is between about 1 mg and about 15 mg promethazine. In some embodiments, the dose delivered transdermally into the blood by a unit dose pack of the invention is 12.5 mg promethazine. In some embodiments, the dose delivered transdermally into the blood by a unit dose pack of the invention is 6.25 mg. In some embodiments, the dose delivered transdermally into the blood by a unit dose pack of the invention is 3.13 mg. In some embodiments, the dose delivered transdermally into the blood by a unit dose pack of the invention is 1.56 mg. In some embodiments, the dose delivered transdermally into the blood by a unit dose pack of the invention is 1.0 mg. Thus, the unit dosage pack of the invention provides specific single dosages of promethazine for transdermal administration. The selection the concentration of drug in the unit dose pack of the invention is designed to provide the administration of therapeutically effective amounts of promethazine for the treatment of PONV, pain, and pruritus while minimizing the risk of transdermal administration of an amount of promethazine which can cause undesirable side effects. Such selection is made by trained medical personnel and is guided by medical parameters known to those with skill in the art. In one embodiment, a unit dose pack may contain less than about 100 mg of promethazine. In another embodiment, the unit dose pack of the invention may contain about 1 mg promethazine to about 80 mg promethazine. In still another embodiment, the unit dose pack of the invention may contain between 20 mg promethazine to about 50 mg promethazine.

One non-limiting example of a unit dosage device that can be used include the SNAP® unit dose pack (Tapemark, Inc., St. Paul, Minn.). Another example of a unit dosage device which is useful in the invention is DelPouch® (Catelent Pharma Solutions, Somerset, N.J.)

Formulations of the invention are substantially free of the components of pluronic lecithin organogel (PLO). Combinations of isopropyl palmitate, soy lecithin, and Pluronic F-127™ in concentrations known to form pluronic lecithin organogel are absent in formulations of the invention. Other embodiments of the invention may employ other gel compositions, formulated as described herein.

In some embodiments, formulations of the invention may be delivered through the use of a transdermal patch. In such embodiments, an object of the invention, that of controlling a single topically applied transdermal dose of promethazine, is achieved by adjusting parameters including size of patch or coverage area, concentration of the drug, duration of therapeutic drug level, and use of a transdermal penetration enhancer.

In some embodiments, formulations of the invention may be delivered through the use of a transdermal device. In such embodiments, an object of the invention, that of controlling a single topically applied transdermal dose of promethazine, is achieved by adjusting parameters including size of the coverage area, concentration of the drug, volume of drug reservoir, duration of therapeutic drug level, and use of a transdermal penetration enhancer.

The topically applied transdermal formulation is particularly useful to treat post-operative patients, including those released or remaining in the hospital setting. The topically applied transdermal formulation is particularly useful in the prevention and/or treatment and control of nausea and vomiting occurring before, during, or up to 72 hours after surgical procedures or diagnostic procedures in adults or children over the age of two years; in the prevention and/or treatment and control of nausea and vomiting occurring before, during, or up to 72 hours after general anesthesia in adults or children over the age of two years; in the prevention and/or treatment and control of nausea and vomiting occurring before, during, or up to 72 hours after regional anesthesia in adults or children over the age of two years; and in the prevention and/or treatment and control of nausea and vomiting occurring before, during, or up to 72 hours after local anesthesia in adults or children over the age of two years.

The topically applied transdermal formulation may also useful in the prevention and/or treatment of allergic conditions in adults or children over the age of two years; in the prevention and/or treatment of anaphylaxis in adults or children over the age of two years; in the prevention and/or treatment and control of nausea and vomiting occurring before, during or up to 72 hours after physical motion in adults or children over the age of two years; and in the prevention and treatment and control of nausea and vomiting associated with disorders of the vestibular apparatus in adults or children over the age of two years. Moreover, the topically applied transdermal formulation may also useful in the prevention and treatment and control of nausea and vomiting associated with chemotherapy and radiotherapy for cancer or leukemia in adults or children over the age of two years; in combination with an opiate for the prevention and treatment of pain; and, in the prevention and treatment and control of nausea and vomiting associated with drug therapy in adults or children over the age of two years. Additionally, the topically applied transdermal formulation may also useful in the prevention and treatment and control of nausea and vomiting associated with migraine in adults or children over the age of two years; in the prevention and treatment and control of nausea and vomiting associated with epilepsy in adults or children over the age of two years; in the prevention and treatment and control of nausea and vomiting associated with cancer or leukemia in adults or children over the age of two years; in the prevention and treatment and control of nausea and vomiting associated with food poisoning in adults or children over the age of two years; in the prevention and/or treatment and control of nausea and vomiting occurring during the first and second trimesters of pregnancy; in the prevention and/or treatment and control of hyperemesis gravidarium; and, in the prevention and/or treatment and control of nausea and vomiting that can lead to inhalation of stomach contents.

Formulations of the invention are useful in, but such usefulness is not limited to:

The provision of adjunctive anti-emetic effects following or with other anti-emetic therapy before, during, or up to 72 hours after surgical or diagnostic procedures in adults or children over the age of two years;

The provision of adjunctive analgesic effects following or with other analgesic therapy before, during, or up to 72 hours after surgical or diagnostic procedures in adults or children over the age of two years;

The provision of adjunctive anti-cholinergic effects following or with other anti-cholinergic therapy before, during, or up to 72 hours after surgical or diagnostic procedures in adults or children over the age of two years;

The provision of adjunctive sedative effects following or with other sedative therapy before, during or up to 72 hours after surgical or diagnostic procedures in adults or children over the age of two years; and

The provision of adjunctive anxiolytic effects following or with other anxiolytic therapy before, during, or up to 72 hours after surgical or diagnostic procedures in adults or children over the age of two years.

The prevention and/or treatment and control of apprehension occurring before, during, or up to 72 hours after surgical procedures or diagnostic procedures in adults or children over the age of two years;

The prevention and/or treatment of dermographism in adults or children over the age of two years;

The prevention of Mendelssohn's Syndrome;

The production of a light sedation in adults or children over the age of two years; in the production of pain relief in adults or children over the age of two years;

The production of pain relief before, during, or after labor;

The assistance of recovery from local, regional or general anesthesia in adults or children over the age of two years;

The stimulation of appetite in adults and children over the age of two years;

The treatment of pain when applied concurrently with buprenorphine therapy;

The treatment of opiate and cocaine addiction when applied concurrently with buprenorphine therapy;

The treatment of opiate and cocaine addiction when applied concurrently with buprenorphine and naloxone therapy;

The prevention, treatment, and control of chemotherapy-induced nausea and vomiting;

The prevention, treatment, and control of radiotherapy-induced nausea and/or vomiting;

The prevention, treatment, and control of hyperemesis gravidarium;

The prevention, treatment, and control of nausea and vomiting of pregnancy in first, second and third trimesters;

The prevention, treatment, and control of nausea and vomiting associated with delivery and obstetric procedures;

The prevention, treatment, and control of motion sickness;

The prevention, treatment, and control of allergic conditions, including, but not limited to, perennial and seasonal allergic rhinitis, vasomotor rhinitis, allergic conjunctivitis due to inhalant allergens and foods, mild, uncomplicated allergic skin manifestations of urticaria and angioedema, and amelioration of allergic reactions to blood or plasma;

The control of anaphylactic reactions, including, but not limited to adjunctive therapy to epinephrine or other standard treatment modalities;

The provision of sedation;

The relief of apprehension and production of light sleep from which the patient can be easily aroused, including, but not limited to preoperative, postoperative, or obstetric sedation.

The treatment of motion sickness, nausea and vomiting associated with anesthesia and surgery, perennial and seasonal allergic rhinitis, vasomotor rhinitis, allergic conjunctivitis due to inhalant allergens and foods, mild, uncomplicated allergic skin manifestations of urticaria and angiodema, allergic reactions to blood or plasma, dermographism.

The conditions of chemotherapy induced nausea and vomiting and radiotherapy induced nausea and vomiting, nausea and vomiting of pregnancy, and hyperemesis gravidarium are characterized by nausea, retching and vomiting. These are coordinated by the vomiting center in the lateral medullary reticular formation in the pons. Chemicals in the cerebrospinal fluid and blood can have a direct stimulating effect at the vomiting center mediated through dopaminergic, cholinergic and histaminic neurochemical receptor sites. These pathways are antagonized by promethazine. Compositions of the invention block these neurochemical receptor sites by providing transdermal promethazine, thus treating or preventing these conditions.

The following examples are provided to be illustrative of some embodiments of the invention and are not limiting. It is recognized that alternative components, temperatures, ratios of components, pH, mixing methods, mixing times, isolation procedures and other recognized formulation parameters are well-known to those skilled in the art, and are within the scope of the present invention.

### EXAMPLE 1

Aqueous Phase Preparation: In a suitable container, promethazine and propylene glycol are dissolved in 10-30 ml purified water at room temperature. Oil Phase Preparation: In a suitable container, white petrolatum, isopropyl myristate, Pemulen TR-2, Carpool 981, methylparaben, and propylparapen are added together, heated to 50-60° C. and mixed thoroughly. 5% Promethazine Cream Preparation: Once the oil phase is melted and mixed, the aqueous phase is added slowly under continuous mixing. After mixing the two phases, phosphate buffer is added to adjust pH to 7.0. Finally water is added to complete the desired volume.

| EXCIPIENT | % W/W | FUNCTION |
|---|---|---|
| Promethazine | 5.0 | Active Pharmaceutical Ingredient |
| Isopropyl Myristate | 1.0-25.0 | Oil Phase/Surfactant |
| White Petrolatum | 1-10 | Oil Phase |
| Propylene Glycol | 1-10 | Solvent/Emulsifying agent |
| Pemulen | 0.05-5.0 | Emulsifying agent |
| Carpool 981 | TR-2 0.1-5.0 | Emulsion stabilizer |
| Methylparaben | 0.05-0.5 | Preservative |
| Propylparaben | 0.05-0.5 | Preservative |
| Phosphate Buffer | Qs pH ~7.0 | pH Modulator |

### EXAMPLE 2

Lecithin/isopropyl myristate solution: In a glass container, Lecithin is added to sorbic acid and mixed thoroughly with isopropyl myristate. The mixture is covered to complete dissolution for 3 to 8 hours. Pluro Gel 30% (Poloxamer 407): potassium sorbate is dissolved in 50 ml of cool H₂O. Poloxamer 407, methylparaben, and propylparaben are added and mixed and the volume is completed to 100 ml with water. The gel is covered and refrigerated to allow dissolution for 12 to 24 hours. Promethazine 5% gel (50 mg/ml): Promethazine is dissolved in H₂O (1 ml H₂O/1 g promethazine). The promethazine solution is thoroughly dissolved in 23 ml of LEIP solution and blended. The resulting solution is later diluted to 100 ml with Pluro gel until the desired thickness is reached.

| EXCIPIENT | % W/W | FUNCTION |
|---|---|---|
| Promethazine | 5.0 | Active Pharmaceutical Ingredient |
| Lecithin | 1.0-25.0 | Oil Phase/Surfactant |
| Sorbic acid | 0.05-1.0 | Preservative |
| Isopropyl | 1.0-25.0 | Thickening Agent |
| Pemulen | 0.05-5.0 | Emulsifying agent |
| Poloxamer 407 | 10-40 | Oil Phase/Surfactant |
| Potassium Sorbate | 0.05-5.0 | Preservative |
| Methylparaben | 0.05-0.5 | Preservative |
| Propylparaben | 0.05-0.5 | Preservative |
| Water | q.s. ad 100 | Aqueous Phase |

### EXAMPLE 3

Promethazine liposomes are prepared by injecting solubilized lipid (sphingomyelin-cholesterol at 63/37 [mol/mol]) into 0.5 M H₂SO4 and extruding with a LIPEX extruder (Northern Lipids Inc., Vancouver, Canada). Vesicles are diluted in 115 mM NaSO4-50 mM NaH2PO4, and the external pH is adjusted to 7.5. Promethazine is added at a drug/lipid ratio of 1:3 (mol/mol) and loaded at 60° C. The formulation is then diafiltered (Midgee ultrafiltration column; Amersham, Piscataway, N.J.) to remove unencapsulated drug and solvent and concentrated to 100 mg of lipid/ml.

### EXAMPLE 4

Promethazine 5% liposomes are prepared by injecting solubilized lipid (dipalmatoylphosphatidylethanolamine (DPPE): cholesterylhemisuccinate (CHEMS) into 0.5 M H2SO4 and extruding with a LIPEX extruder (Northern Lipids Inc., Vancouver, Canada). Vesicles are diluted in 115 mM NaSO4-50 mM NaH2PO4, and the external pH is adjusted to 7.5. Promethazine is added at a drug/lipid ratio of 1:3 (mol/mol) and loaded at 60° C. Drug : DPPE : CHEMS is 5%:57%:37% w/w. The formulation is then diafiltered (Midgee ultrafiltration column; Amersham, Piscataway, N.J.) to remove unencapsulated drug and solvent and concentrated to 100 mg of lipid/ml.

All documents cited herein are incorporated by reference in their entirety for all purposes.

We claim:

1. A topical formulation comprised of an emulsifying agent, an emulsion stabilizer, a preservative, a permeation enhancer and promethazine.

2. The formulation of claim 1 containing from about 1% to about 5% promethazine.

3. A formulation comprising promethazine, isopropyl myristate, white petrolatum, propylene glycol, Pemulen, Carbopol 981, methyl paraben, propyl paraben, and phosphate buffer.

4. The formulation of claim 3 comprising about 1% to 5% promethazine.

5. The formulation of claim 3 comprising about 5% promethazine.

6. A liposomal formulation comprising about 1% to about 5% promethazine.

7. The formulation of claim 6 comprising about 5% promethazine.

8. A unit dose pack comprising the formulation of claim 4, wherein the unit dose pack comprises less than about 100 mg of promethazine.

9. The unit dose pack of claim 8 comprising about 1 mg to about 80 mg promethazine.

10. The unit dose pack of claim 9 comprising about 20 mg promethazine to about 50 mg promethazine.

11. The unit dose pack of claim 8 wherein the concentration of promethazine in the unit dose pack delivers a transdermal dose of a mass selected from the set consisting of 1mg, 1.56 mg, 3.13 mg, 6.25 mg, and 12.5 mg.

12. A unit dose pack comprising the formulation of claim 6, wherein the unit dose pack comprises less than about 100 mg of promethazine.

13. The unit dose pack of claim 12 comprising about 1 mg promethazine to about 80 mg promethazine.

14. The unit dose pack of claim 12 comprising about 20 mg promethazine to about 50 mg promethazine.

15. The unit dose pack of claim 8 wherein the concentration of promethazine in the unit dose pack delivers a transdermal dose of a mass selected from the set consisting of 1mg, 1.56 mg, 3.13 mg, 6.25 mg, and 12.5 mg.

16. A method of treating or preventing post-operative nausea and vomiting comprising applying formulation of claim 4 to the skin of a patient in need thereof.

17. A method of treating or preventing post-operative nausea and vomiting comprising applying formulation of claim 6 to the skin of a patient in need thereof.

18. A method of prevention, treatment, and/or control of a condition comprising applying the formulation of claim 4 to the skin of a patient in need thereof, said condition being selected from the group consisting of chemotherapy-induced nausea and vomiting, radiotherapy-induced nausea and vomiting, hyperemesis gravidarium, nausea and vomiting of pregnancy in the first, second or third trimesters, nausea and vomiting associated with childbirth and obstetric procedures, motion sickness, nausea and vomiting associated with anesthesia and surgery, perennial and seasonal allergic rhinitis, vasomotor rhinitis, allergic conjunctivitis due to inhalant allergens and foods, mild, uncomplicated allergic skin manifestations of urticaria and angiodema, allergic reactions to blood or plasma, dermographism, anaphylactic reactions, pain, sedation, apprehension, and pruritus.

19. A method of prevention, treatment, and/or control of a condition comprising applying the formulation of claim 6 to the skin of a patient in need thereof, said condition being selected from the group consisting of chemotherapy-induced nausea and vomiting, radiotherapy-induced nausea and vomiting, hyperemesis gravidarium, nausea and vomiting of pregnancy in the first, second or third trimesters, nausea and vomiting associated with childbirth and obstetric procedures, motion sickness, nausea and vomiting associated with anesthesia and surgery, perennial and seasonal allergic rhinitis, vasomotor rhinitis, allergic conjunctivitis due to inhalant allergens and foods, mild, uncomplicated allergic skin manifestations of urticaria and angiodema, allergic reactions to blood or plasma, dermographism, anaphylactic reactions, pain, sedation, apprehension, and pruritus.
